(19) United States
(12) Patent Application Publication
(10) Pub. No.: US 2020/0231838 A1
(43) Pub. Date: Jul. 23, 2020

(54) SELF ASSEMBLING MIXED BLOCK COPOLYMER FOR NANOSTRUCTURED FUNCTIONAL FILMS

(71) Applicant: Her Majesty the Queen in Right of Canada, as Represented by the Minister of Agriculture and Agri-Foo, Saint-Hyacinthe (CA)

(72) Inventors: Louise DESCHÊNES, Saint-Hyacinthe (CA); Béatrice LEGO, Montréal (CA); François SAINT-GERMAIN, Saint-Hyacinthe (CA); Normand ROBERT, Saint-Hyacinthe (CA)

(21) Appl. No.: 16/483,160

(22) PCT Filed: Feb. 6, 2018
(86) PCT No.: PCT/CA2018/050134

§ 371 Date: Aug. 2, 2019

Related U.S. Application Data
(60) Provisional application No. 62/455,358, filed on Feb. 6, 2017.

(51) Int. Cl. C09D 153/02; G01N 33/545; G01N 21/59; C09D 153/00

(52) U.S. Cl. CPC C09D 153/025; G01N 33/545; B82Y 30/00; C09D 153/005; G01N 21/59

(57) ABSTRACT

Functionalizable nanopatterned monolayers comprise one or more block copolymers, each containing one or more hydrophobic blocks and one or more hydrophilic blocks. The one or more hydrophilic blocks of at least one of the block copolymers can be terminated by a modifiable functional group, to which a functional moiety, such as a biological molecule, can be attached. The surface concentration of the modifiable functional groups on the monolayer can be controlled by adjusting the properties of the block copolymers, such as their size, their chemical makeup, and the relative proportion of the block copolymer containing the modifiable functional group, and the conditions, such as surface pressure, under which the monolayer is formed and/or transferred to a substrate. The nanopatterned monolayer can be transferred to a substrate to form a functionalizable nanopatterned nanocoating, which is useful in applications such as biosensors.

### BACKGROUND

The present application is directed to a functionalizable nanofilm and methods of preparing the functionalizable nanofilm. More specifically, the present application is directed to a functionalizable nanofilm or nanocoating formed from one or more block copolymers and to methods of preparing and functionalizing the functionalizable nanofilm or nanocoating.

The use of nanofilms (ultra-thin films comprising one or a few layers of atoms or molecules in thickness) to form nanocoatings on surfaces has found application in a wide range of industries. For example, anti-microbial nanocoatings are used in applications such as health care, water treatment equipment, food manufacturing, and packaging. Anti-fouling and easy-to-clean nanocoatings are used in the marine, food manufacturing, automotive, and electronics industries, among others. The use of anti-fingerprint nanocoatings is projected to grow in the electronics, automotive, medical and healthcare industries.

The functional properties of a nanocoating on a surface can result from the physicochemical properties of the material of the nanocoating, such as hydrophilicity or electrical charge. In some cases, nanocoatings may also bear molecules or groups which have a specific chemical or biological functionality. Such specific functionality can include, for example, the ability to carry out or to catalyze a specific chemical reaction, or to undergo specific recognition or binding by another moiety. In such cases, the ability to precisely control features such as the positioning, orientation and surface concentration of functional molecules or groups on a nanocoating can be advantageous, so as to more finely adjust the functional properties of the coated surface.

Organic thin films, including nanofilms, can be deposited on solid substrates to form nanocoatings by many techniques, including thermal evaporation, sputtering, electrodeposition, molecular beam epitaxy, adsorption from solution, molecular self-assembly, Langmuir-Blodgett/Schaefer methods and others. However, known methods of producing nanofilms may not provide precise control of the functional properties of the nanofilm, including the positioning, orientation, and surface concentration of functional molecules or groups on the nanofilm or on a nanocoating formed by deposit of the nanofilm on a surface. New methods of producing nanofilms which allow improved control of the functionalization of the nanofilms are therefore desirable.

### SUMMARY

The present application provides a method of preparing a functionalizable nanopatterned nanocoating. The method includes forming a nanopatterned nanofilm comprising one or more block copolymers, wherein each of the one or more block copolymers comprises one or more hydrophobic blocks and one or more hydrophilic blocks, and at least one of the one or more block copolymers comprises at least one hydrophilic block which is terminated by a modifiable functional group. The method further includes coating a substrate with the nanopatterned nanofilm to form the functionalizable nanopatterned nanocoating. In at least one embodiment, coating the substrate with the nanopatterned nanofilm includes transferring the nanopatterned nanofilm to the substrate to form the functionalizable nanopatterned nanocoating. In at least one embodiment, coating the substrate with the nanopatterned nanofilm includes adsorbing the nanopatterned nanofilm on the substrate to form the functionalizable nanopatterned nanocoating.

In at least one embodiment, the method further includes functionalizing the functionalizable nanopatterned nanocoating with a functional moiety to provide a functionalized nanopatterned nanocoating. In at least one embodiment the functional moiety is a biological molecule. In at least one embodiment, the biological molecule is a recombinant biological molecule.

In a further aspect, the present application provides a functionalizable nanopatterned nanocoating prepared as described herein. Yet a further aspect of the present application provides a functionalized nanocoating prepared as described herein.

Another aspect of the present application provides a sensor including a sensor surface coated with a functionalized nanocoating as described herein. In at least one embodiment, the sensor is a biosensor.

### BRIEF DESCRIPTION OF THE DRAWINGS

Further features of the present invention will become apparent from the following written description and the accompanying figures, in which:

FIG. 1 is a diagrammatic representation of a monolayer of micelles or nanodomains of an amphiphilic block copolymer formed at an air-water interface at a surface concentration allowing for polymer brush formation;

FIG. 2 is a diagrammatic representation of a nanopatterned monolayer of the present amphiphilic block copolymer at an air-water interface;

FIG. 3 is a diagrammatic representation of a nanocoating prepared by transferring the nanopatterned monolayer of FIG. 2 to a surface, and including an additional component to prevent non-specific adsorption to the surface;

FIG. 4A is an atomic force microscopy (AFM) image obtained on a Bruker Multimode 8 atomic force microscope with an ARROW™-NCR-W probe from Nanoworld (k=42 N/m) (scale 5 μm×5 μm), of the block copolymer PS₁₈₃-PEO₂₈₀ spread at an air-water interface (1 mg/ml solution in chloroform) and transferred onto a polystyrene substrate at a surface pressure of 2 mN/m;

FIG. 4B is an AFM image (scale 1 μm×1 μm) obtained with ARROW NCR-W probes with a frequency of 285 kHz and a spring constant of 42 N/m of the block copolymer PS₁₈₃-PEO₂₈₀ spread at an air-water interface (1 mg/ml solution in chloroform) and transferred at a surface pressure of 2 mN/m onto a hydrogen-passivated silicon (Si—H) hydrophobic surface;

FIGS. 4C to 4E are AFM images (scale 1 μm×1 μm) of the block copolymer PS₁₈₃-PEO₂₈₀ spread at an air-water interface (1 mg/ml solution in chloroform) and transferred onto a Si—H surface at surface pressures of 2 mN/m (4C), 8.5 mN/m (4D) and 11 mN/m (4E), respectively;

FIG. 4F is an AFM image (scale 1 μm×1 μm) of the block copolymer PS₁₈₃-PEO₂₈₀ spread at an air-water interface (1 mg/ml solution in chloroform) and transferred to a XanTec SPR gold chip at a surface pressure of 11 mN/m.;

FIG. 5 is a diagrammatic representation of an experimental set-up used to functionalize a surface plasmon resonance (SPR) gold chip coated with an embodiment of the present functionalizable nanopatterned nanocoating prepared from the block copolymers PS₁₈₃-PEO₂₈₀ and PS₁₈₃-PEO₂₈₀-maleimide with a functional moiety (cysteine-tagged protein G);

FIG. 6 is a graph showing surface plasmon resonance (SPR) sensorgrams obtained when an uncoated Au surface and an Au surface previously exposed to EO₇—S—S-EO₇ are each exposed to cysteine-tagged protein G. Addition of cysteine-tagged protein G is indicated at the arrow;

FIG. 7 is a graph showing an SPR sensorgram obtained when a gold surface coated with an embodiment of the present functionalizable nanopatterned nanofilm prepared from the block copolymer PS₁₈₃-PEO₂₈₀ containing 10% PS₁₈₃-PEO₂₈₀-maleimide is exposed to cysteine-tagged protein G;

FIG. 8 is a graph showing an SPR sensorgram obtained when an SPR chip coated with an embodiment of the present nanopatterned nanocoating functionalized with protein G is exposed to a preparation of immunoglobulin G (IgG), then further exposed to glycine/HCl solution at pH 1.5; and

FIG. 9 is a bar graph showing the response seen when an SPR chip coated with an embodiment of the present nanopatterned nanocoating functionalized with protein G is repeatedly alternately exposed to phosphate-buffered saline (PBS) containing IgG at concentrations of either 0.01 mg/mL or 0.1 mg/mL and cleaned between exposures.

### DETAILED DESCRIPTION

The present application provides a method of preparing a functionalizable nanopatterned nanocoating including forming a functionalizable nanopatterned nanofilm and coating a substrate with the nanofilm to form the nanocoating. As used herein, the term “nanofilm” is intended to mean a film or layer of material having a thickness in the nanoscale (ranging from about 1 nm to about 100 nm), as defined in International Organization for Standardization (ISO) standards ISO/TR 18401:2017 (Nanotechnologies—Plain language explanation of selected terms from the ISO/IEC 80004 series) and ISO/TS 80004-11:2017(E) (Nanotechnologies—Vocabulary, Part 11: Nanolayer, nanocoating, nanofilm and related terms). In at least one embodiment, a nanofilm can be composed of one or more monolayers. As used herein, the term “monolayer” is intended to mean a layer of atoms or molecules that is one atom or molecule in thickness. As used herein, the term “nanocoating” is intended to mean a coating formed when a nanofilm is adsorbed, deposited, transferred or otherwise coated onto a surface.

The nanofilm comprises one or more block copolymers. As used herein, the term “block copolymer” is intended to mean a polymeric molecule comprising two or more polymeric segments or blocks covalently bonded together. At least one of the two or more polymeric blocks is formed by polymerization of a monomer which is different from the monomer from which at least one other of the two or more polymeric blocks is formed by polymerization. Thus, as is understood in the art, block copolymers are characterized by covalent bonding between polymeric blocks respectively made by polymerizing different monomers.

In at least one embodiment, the block copolymers are amphiphilic block copolymers comprising one or more hydrophobic (Pβ) blocks and one or more hydrophilic (Pλ) blocks. In at least one embodiment, the hydrophobic blocks and the hydrophilic blocks are immiscible. In at least one embodiment, the hydrophilic block is a surface-active hydrophilic block. In at least one embodiment, the polymer comprising the hydrophobic block is polystyrene (PS; —[—CH₂—CH(C₆H₅)—]ₓ—) or polybutadiene (PB; —[—CH₂—CH═CH—CH₂]ₓ). In at least one embodiment, the polymer comprising the hydrophilic block is polyethylene oxide (PEO; —[—CH₂—CH₂—O—]_{y}—H). Persons skilled in the art would be aware of other polymers suitable for each of the hydrophobic and hydrophilic blocks in light of the teaching herein. In at least one embodiment, a hydrophobic block can be bonded to a single hydrophilic block. In at least one embodiment, a hydrophobic block can be bonded to two or more hydrophilic blocks.

Thus, examples of block copolymers suitable for the present invention include but are not limited to polystyrene-polyethylene oxide (PSₓ-PEO_{y}), polybutadiene-polyethylene oxide (PBₓ-PEO_{y}), polyethylene oxide-polystyrene-polyethylene oxide (PEO_{y}-PSₓ-PEO_{y}) and polyethylene oxide-polybutadiene-polyethylene oxide (PEO_{y}-PBₓ-PEO_{y}), where x represents the number of styrene or butadiene monomers per hydrophobic block and y represents the number of ethylene oxide monomers per hydrophilic block of the block copolymers. Thus, for example, polystyrene-polyethylene oxide (PSₓ-PEO_{y}) can be represented by the following structural formula:

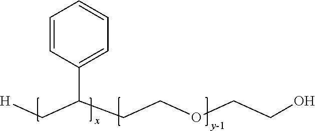

and polyethylene oxide-polystyrene-polyethylene oxide (PEO_{y}-PSₓ-PEO_{y}) can be represented by the following structural formula:

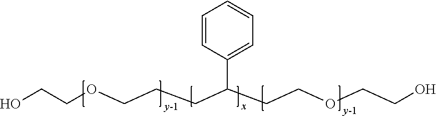

In at least one embodiment, the ratio between x and y is from about 0.3:1 to about 2:1. In at least one embodiment, the percentage by weight of the hydrophilic polymer in the block copolymer is from about 15% to about 60%.

In at least one embodiment, such amphiphilic block copolymers can self-assemble to form nanopatterned nanofilms. As used herein in the context of a nanopatterned nanofilm or nanocoating, the term “nanopatterned” or “nanostructured” is intended to mean that the block copolymers which make up the nanofilm or nanocoating can self-assemble to form locally aggregated structures, or nanodomains, which are distributed within the nanofilm or nanocoating to form a pattern on a nanoscale.

Without being bound by theory, it is readily understood by the skilled person that a hydrophilic polymeric block will have a higher affinity for water than will a hydrophobic polymeric block. In addition, a hydrophilic polymeric block will tend to associate more with other hydrophilic blocks than with hydrophobic blocks, and a hydrophobic polymeric block will tend to associate more with other hydrophobic blocks than with hydrophilic blocks. Thus, in aqueous solution, such amphiphilic block copolymers may aggregate to form micelles, in which the hydrophilic blocks of a number of block copolymer molecules surround a core containing the hydrophobic blocks of the molecules. In this way, the hydrophilic blocks can interact with the surrounding water molecules, while the hydrophobic blocks can preferentially associate with each other within the core of the micelle, reducing the less favourable interaction between the hydrophobic blocks and the water phase.

Similarly, at an air-water interface, such amphiphilic block copolymers can form a monolayer, or Langmuir film. As used herein, the term “air-water interface” is intended to mean interface between air and an aqueous subphase, including but not limited to pure water or a solution of one or more solutes in water. In at least one embodiment, a Langmuir film can be prepared by spreading a solution of one or more amphiphilic block copolymers at an air-water interface. The spreading solution may be formed by dissolving the amphiphilic block copolymer in a spreading solvent. In at least one embodiment, the spreading solvent has a low solubility in water. In at least one embodiment, the spreading solvent may also have a relatively high vapour pressure, so that it is relatively volatile and can evaporate from the air-water interface, such that only the amphiphilic block copolymer molecules remain as a monolayer at the air-water interface. Suitable spreading solvents are well known in the art and include, but are not limited to, chloroform (CHCl₃).

Monolayers of amphiphilic block copolymers containing a surface-active hydrophilic block which is immiscible with the hydrophobic block can form micelles or other locally aggregated structures, referred to herein as nanodomains, at an air-water interface. Thus, when a solution of such an amphiphilic block copolymer in a spreading solvent is spread on an air-water interface, the block copolymer molecules can self-assemble to form a pattern within the resulting monolayer. The specific pattern formed depends on several factors, including the composition of the copolymer and its architecture, the concentration of copolymer, and the spreading conditions, including but not limited to the spreading solvent, temperature, composition of the aqueous phase and other conditions well known in the art.

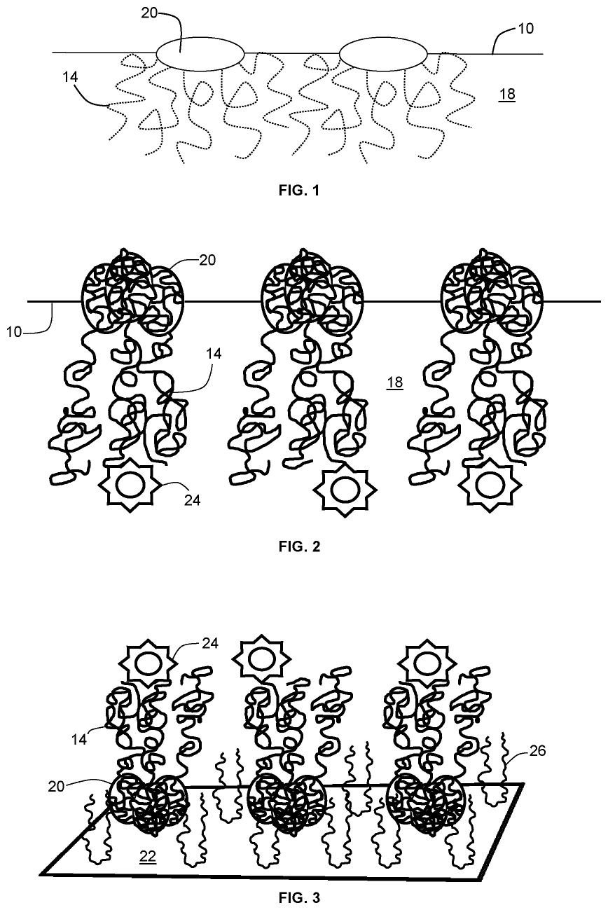

Models for the formation of micelles at an air-water interface in a monolayer of an amphiphilic block copolymer have been proposed in the literature. Without being bound by theory, it is believed that, upon spreading a solution of an amphiphilic block copolymer in a spreading solvent at an air-water interface, the hydrophobic block is preferentially concentrated in the less polar solvent phase while the hydrophilic block can interact more favourably with the aqueous subphase and tends to spread at the interface between the solvent phase and the aqueous subphase. As the solvent phase evaporates, the copolymer molecules can self-assemble to form micelles. The hydrophobic blocks can aggregate to form a hydrophobic subdomain 20 which is oriented towards the air phase and which anchors the hydrophilic blocks 14 at the air-water interface 10, as seen in FIG. 1 (Deschênes et al, Langmuir (2008), 24: 3699-3708).

Again without being bound by theory, it is believed that as the solvent phase evaporates, the formation of micelles occurs at the interface between the solvent phase, the aqueous subphase and the surrounding air. Furthermore, the local concentration of the block copolymer molecules, and thus the size, shape and distribution of micelles formed at the interface between the solvent phase, the aqueous subphase and the surrounding air, is affected by the rate of evaporation of the solvent phase and the degree to which the solvent phase spreads over the aqueous subphase.

The structure of Langmuir films can be further manipulated by adjusting the conditions under which the monolayer is formed. Reducing the surface area available to the monolayer, for example, by sweeping a barrier across the surface, increases the surface pressure, causing the molecules to approach each other more closely. This can trigger a series of phase transitions that can transform the morphology of the nanodomains. Thus, as the surface concentration increases, a significant portion of the hydrophilic block 14 can be transferred into the aqueous subphase 18. At a sufficient surface concentration and corresponding surface pressure, which will be readily determined by the person of skill in the art in light of the teaching herein, the hydrophilic blocks 14 can form a brush-like structure, or polymer brush, within the aqueous subphase 18, as seen in FIG. 1.

The present amphiphilic block copolymers can thus self-assemble into micelles or nanodomains at an air-water interface 10, as diagrammatically shown in FIG. 2, to form a nanopatterned monolayer or nanofilm. Without being bound by theory, it is believed that the pattern of these nanodomains, including but not limited to the relative size and spacing of the nanodomains within the nanofilm, is determined by the balance of physical and chemical properties between the respective hydrophilic and hydrophobic blocks of the constituent block copolymers. Such chemical and physical properties of the blocks include but are not limited to hydrophobicity or hydrophilicity, polarity, charge, size and conformational flexibility. Other factors, including but not limited to surface pressure, the nature of the spreading solvent, the concentration of the block copolymer in the spreading solution, the properties of the aqueous subphase, including but not limited to pH and the identity and concentration of any electrolytes present, and other conditions under which the nanofilm is formed can also affect the arrangement of the molecules of the amphiphilic block copolymer within the resulting monolayer. The effects of such factors on the formation of nanodomains in nanopatterned nanofilms formed from amphiphilic block copolymers have been discussed in several research papers and reviews, including but not limited to:
(1) Kim and Kim, 2017, Effective morphology control of block copolymers and spreading area-dependent phase diagram at the air/water interface, J. Phys. Chem. Lett., 8, 1865-1871;
(2) Kita-Tokarczyk, K., Junginger, M., Belegrinou, S and Taubert, A. 2011, Amphiphilic Polymers at Interfaces, Adv Polym Sci (2011) 242: 151-201; and
(3) Park, J. Y. and Advincula, R. C., 2011, Nanostructuring polymers, colloids, and nanomaterials at the air-water interface through Langmuir and Langmuir-Blodgett techniques, Soft Matter, 7, 9829.

Addition of small molecules can also be used to modify and/or control the morphology of block copolymer monolayer films at the air-water interface, as described in Perepichka, 1.1, Chen, X. and Bazuin, G., 2013, Nanopatterning of substrates by self-assembly in supramolecular block copolymer monolayer films, Sci China Chem January (2013) Vol. 56 No. 1.

Therefore, advantageously, polymers suitable for the hydrophobic block of the block copolymer exhibit reduced or minimal spreading at an air-water interface, so as to readily form nanodomains upon evaporation of a spreading solvent. In addition, polymers suitable for the hydrophobic block are advantageously selected to be easily dissolved in volatile solvents which can be readily removed by evaporation and used as spreading solvents. Advantageously, polymers suitable for the hydrophilic block of the block copolymer are surface-active and can adsorb at an air-water interface. Furthermore, advantageously, polymers suitable for the hydrophilic block of the block copolymer can be transferred into the aqueous subphase of the air-water interface upon compression.

Advantageously, the balance between the lengths of the hydrophobic block and the hydrophilic block allows the hydrophobic block to form hydrophobic subdomains which can act to anchor the block copolymer at an air-water interface, while the hydrophilic block can provide sufficient coverage to form a polymer brush in the aqueous subphase, thus allowing formation of a nanofilm with a well-defined pattern of nanodomains, and which can be capable of reducing non-specific adsorption, as discussed herein. Thus, in at least one embodiment, the ratio of the number of hydrophobic monomers in the hydrophobic block to the number of hydrophilic monomers in the hydrophilic block in the present block copolymer is from about 0.3:1 to about 2:1. In at least one embodiment, the percentage by weight of the hydrophilic polymer in the block copolymer is from about 15% to about 60%. In at least one embodiment, block copolymers having such a composition can form a nanopatterned nanofilm at an air-water interface in which the hydrophilic blocks form a brush-like structure within the aqueous subphase.

The nanopatterned nanofilm can be deposited on, transferred to or otherwise coated on a solid substrate to form a nanopatterned nanocoating. The solid substrate can be any substrate suitable for coating with a block co-polymer, including but not limited to polystyrene, hydrogen-passivated silicon (Si—H), gold-coated glass plates and other substrates known in the art. Thus, suitable substrates also include but are not limited to substrates suitable for ELISA-type immunoassays or substrates suitable for surface plasmon resonance (SPR) and quartz crystal microbalance (QCM) detectors.

Suitable coating techniques are known in the art, including but not limited to the formation of Langmuir-Blodgett/Schaefer films, dipping the substrate in a solution, spin coating and other methods known in the art for creating thin films of block copolymers having various morphologies and orientation. Suitable methods include but are not limited to those described in Tata et al., 2009, “Control of morphology orientation in thin films of PS-b-PEO diblock copolymers and PS-b-PEO/resorcinol molecular complexes”, European Polymer Journal 45, 2520-2528 and in Huang et al., 2007, “Formation of ordered microphase-separated pattern during spin coating of ABC triblock copolymer”, The Journal of Chemical Physics, 126, 104901).

In at least one embodiment, the nanopatterned nanofilm is deposited on or transferred to the solid substrate to form the nanopatterned nanocoating using Langmuir-Blodgett/Schaefer (LB/LS) methods well known in the art, including but not limited to the Langmuir-Blodgett (LB) method and the Langmuir-Schaefer (LS) method. As is known in the art, the Langmuir-Blodgett method involves vertical deposition of the nanofilm on the substrate, while the Langmuir-Schaefer method involves horizontal deposition of the film on the substrate. Such methods of forming the nanopatterned nanocoating can provide one or more advantages, including but not limited to:
ability to precisely control monolayer surface concentration and thickness;
ability to homogenously deposit a monolayer over a large area;
ability to form nanofilms having multilayer structures with varying layer composition; and
ability to deposit monolayers on a wide range of substrates.

In at least one embodiment, the nanopatterned nanofilm is deposited on or transferred to the solid substrate to form the nanopatterned nanocoating using the Langmuir-Schaefer technique. In at least one embodiment, the transfer or deposit of the nanopatterned nanofilm onto the solid substrate is carried out at surface pressures corresponding to the phase transition of PEO around the plateau of the π-A isotherm, which is about 10 mN/m. However, the person skilled in the art would be readily able to determine and select other suitable surface pressures in light of the teaching herein.

In at least one embodiment, as diagrammatically represented in FIG. 3, when the present nanopatterned nanofilm is deposited on or transferred to a substrate to form the nanopatterned nanocoating, the hydrophobic subdomains 20 can adsorb on the surface 22 of the substrate, while the hydrophilic blocks 14 are exposed for interaction with the exterior environment of the nanocoated surface. In addition, in at least one embodiment, the pattern of the nanodomains in the nanopatterned nanofilm determines or is related to the pattern of the nanodomains in the nanopatterned nanocoating formed as the nanofilm is transferred to the surface. Thus, controlling the pattern of the nanofilm as it is formed can provide control over the pattern of the nanocoating on the surface coated by the nanofilm.

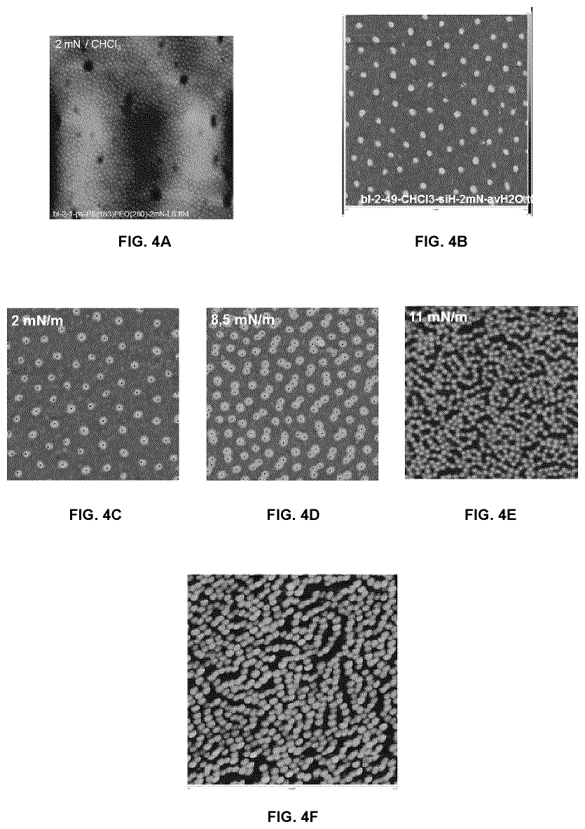

Examples of such nanopatterned nanocoatings prepared by Langmuir-Schaefer transfer of nanofilms formed from the block copolymer PS₁₈₃-PEO₂₈₀ are shown in FIGS. 4A to F. FIG. 4A is an atomic force microscopy (AFM) image showing a regular disposition of nanodomains of a nanocoating of PS₁₈₃-PEO₂₈₀ transferred onto a polystyrene substrate at a surface pressure of 2 mN/m. FIG. 4B shows the size of, and distance between, nanodomains of a nanocoating of PS₁₈₃-PEO₂₈₀ transferred at a surface pressure of 2 mN/m onto a hydrophobic hydrogen-passivated silicon (Si—H) surface. FIGS. 4C to 4E show the size of, and distance between, nanodomains of a nanocoating of PS₁₈₃-PEO₂₈₀ transferred onto a Si—H surface at surface pressures of 2 mN/m, 8.5 mN/m and 11 mN/m, respectively. FIG. 4F shows the size of, and distance between, nanodomains of a nanocoating of PS₁₈₃-PEO₂₈₀ transferred to a XanTec™ surface plasmon resonance (SPR) gold chip at a surface pressure of 11 mN/m.

The present nanopatterned nanofilm or nanocoating is functionalizable. As used herein, the term “functionalizable” is intended to mean that the nanofilm or nanocoating can be readily chemically modified once the nanofilm or nanocoating has formed, so as to provide a functionalized nanofilm or nanocoating. As used herein, the term “functionalized” is intended to mean that the nanofilm or nanocoating bears one or more functional moieties.

As used herein, the term “functional moiety” is intended to mean a group, molecule or group of molecules which can provide a desired functionality to a surface coated by the functionalized nanofilm or nanocoating. Such functionality includes but is not limited to the ability to carry out or catalyze specific chemical reactions or to specifically and/or selectively recognize, bind, capture and/or immobilize other moieties. Thus, in at least one embodiment, a functional moiety can be an organic molecule with a desired three-dimensional (3-D) structure or with a desired chemical reactivity or bioactivity. In at least one embodiment, the organic molecule can be a biomacromolecule, including but not limited to a nucleic acid, such as DNA or RNA, and a protein, including but not limited to enzymes and proteins with specific binding properties such as receptor proteins, antibodies and the like. Those skilled in the art would readily recognize other suitable functional moieties.

At least one of the one or more block copolymers forming the nanofilm or nanocoating comprises at least one hydrophilic block 14 which bears a modifiable functional group 24, as seen in FIGS. 2 and 3. As used herein, the term “modifiable functional group” is intended to mean a group which can be readily chemically modified to attach or form a functional moiety. Thus, in at least one embodiment, a functionalizable nanofilm or nanocoating as described herein can be readily modified to provide a functionalized nanofilm or nanocoating bearing functional moieties formed by chemical modification of the exposed modifiable functional groups.

In at least one embodiment, the modifiable functional group is located at the terminal end of the at least one hydrophilic block. Without being bound by theory, it is conjectured that, when a nanopatterned nanofilm formed as described herein is transferred to a substrate at surface pressures at which the surface coverage of the nanofilm at the air-water interface is sufficiently high, the hydrophilic blocks 14 advantageously form a brush-like structure in the aqueous subphase 18, as diagrammatically illustrated in FIG. 2. Thus, when the resulting nanopatterned nanofilm is coated onto a surface, the terminal ends of the hydrophilic blocks 14 of the nanopatterned nanocoating are exposed to, and can interact with, the external environment of the nanocoated surface, as seen in FIG. 3. Therefore, when the modifiable functional groups are located at the terminal end of at least some of these hydrophilic blocks, the modifiable functional groups can be accessible to the external environment and thus available for chemical modification by any reagents or conditions needed to attach or form a functional moiety.

Therefore, advantageously, polymers suitable for the hydrophilic block of the block copolymer can be readily chemically modified at their terminus to attach or form the modifiable functional group. In at least one embodiment, the block copolymer contains polyethylene oxide (PEO) as at least one of the one or more hydrophilic blocks, and a hydroxyl group (OH) terminating at least one of the PEO blocks can be chemically modified to form the modifiable functional group.

The position, spacing and surface concentration of the modifiable functional groups on a surface bearing the present functionalizable nanopatterned nanocoating can be controlled in several ways. Adjusting the relative concentrations of block copolymer molecules that are unmodified or modified with modifiable functional groups can be used to control the surface concentration of the modifiable functional groups. Furthermore, as discussed above, the balance of physical and chemical properties between the respective hydrophilic and hydrophobic blocks of the constituent block copolymers, including but not limited to the relative lengths of these blocks, and the conditions under which the nanofilm is formed and transferred to a substrate, including but not limited to the selection of spreading solvent and the surface pressure at which the transfer occurs, can control the nanopatterning of the resulting nanofilm. This in turn can affect the positioning and spacing of the modifiable functional groups in three dimensions. Thus, for example, the spacing between modifiable functional groups can be adjusted by adjusting the surface pressure at which the nanofilm is transferred to the substrate. In addition, the size and morphology of the nanodomains within the nanopatterned nanofilm can be adjusted by adjusting the relative lengths of the hydrophobic and hydrophilic blocks of the block copolymer. The presence of molecules in the nanofilm in addition to the block copolymers, including but not limited to homopolymers and small molecules, can further affect the nanopatterning of the resulting nanofilm.

Because the positioning of the modifiable functional groups can be controlled, it is therefore possible to control the positioning and spacing of functional moieties attached to the present functionalized nanofilm or nanocoating, including but not limited to large functional moieties, such as biological molecules or nanoparticles. In this way, mis-orientation or denaturation of attached biomolecules, with resulting loss of desired activity, or steric crowding between functional moieties can be avoided.

In at least one embodiment, the modifiable functional group includes but is not limited to a maleimide or 2-(pyridin-2-yldisulfanyl)ethyl carbamate (2-P2yDSEC) terminal group. Thus, in at least one such embodiment, the one or more block copolymer comprising at least one hydrophilic block which is terminated by a modifiable functional group can be (PS)-(PEO)_{y}-maleimide, represented by the following structural formula:

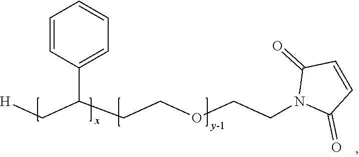

herein abbreviated as

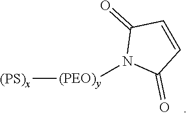

In at least one such embodiment, the one or more block copolymer comprising at least one hydrophilic block which is terminated by a modifiable functional group can be (PS)ₓ-(PEO)_{y}-2-P2yDSEC, represented by the following structural formula:

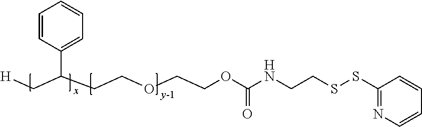

Maleimide and 2-P2yDSEC groups can react with thiols with high specificity. For example, maleimide groups, including but not limited to those attached to a maleimide-modified block copolymer, such as, for example, (PS)ₓ-(PEO)_{y}-maleimide, can react with the thiol (—SH) group of the amino acid cysteine, or of a cysteine tag in a larger biomolecule to form a covalent bond between the maleimide group and the cysteine thiol group. Suitable conditions for this reaction are well known in the art and include but are not limited to an optimal pH of 6.5-7.5 and the presence of a reducing agent, such as tris-(2-carboxyethyl) phosphine (TCEP), which specifically reduces disulfide groups (—S—S—), such as those formed between cysteine residues in some biological molecules to form cystine, to sulfhydryl groups (—SH).

As used herein, the term “cysteine tag” or “cys-tag” is intended to mean a cysteine moiety which is contained in or covalently attached to a molecule, including but not limited to a biological molecule. As used herein, the term “cysteine-tagged” or “cys-tagged” is used to describe a moiety, including but not limited to a biological molecule, which contains or is covalently modified to contain a cysteine tag. The cysteine tag can be part of the amino acid sequence of a protein, including but not limited to a recombinant protein. Alternatively, a biological molecule, including but not limited to a protein or a nucleic acid, can be chemically modified to include a cysteine tag or another thiol group-bearing moiety. Thus, a functionalizable nanofilm or nanocoating containing a maleimide-modified block copolymer, including but not limited to (PS)ₓ-(PEO)_{y}-maleimide, can react with a cysteine tagged functional moiety, including but not limited to a biological molecule or biomolecule, to form a functionalized nanofilm or nanocoating in which the molecule containing the cysteine tag has been covalently attached to the nanofilm or nanocoating, as shown below.

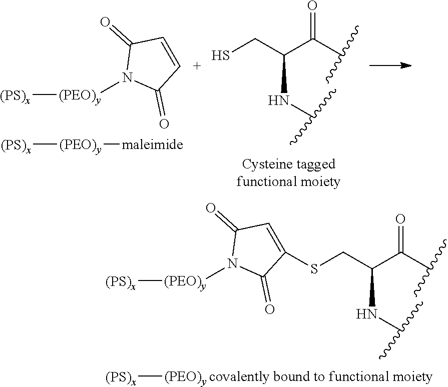

Thus, in at least one embodiment, the functional moiety is a recombinant protein. In at least one embodiment, the functional moiety is a recombinant protein engineered to include one or more cysteine tags.

It will be clear to the person of skill in the art that other modifiable functional groups are possible for the present functionalizable nanopatterned nanofilm or nanocoating, and that other groups present in biomolecules or other functional moieties can be used to attach the biomolecules or other functional moieties to such modifiable functional groups. Such groups are well known in the art, examples of which have been described, for example, in Liu, Y. and Yu, J. 2015. Oriented immobilization of proteins on solid supports for use in biosensors and biochips: a review, Microchim Acta. Thus, the skilled person would be readily able to select modifiable functional groups which could be used to react with such groups, and would be aware of methods which could be used to carry out such reactions.

In at least one embodiment, one or more functional moieties bound to the functionalized nanopatterned nanofilm or nanocoating are selected to selectively recognize, bind, capture and/or immobilize other moieties. In such embodiments, it may be desired to avoid or reduce non-specific adsorption to the functionalized nanopatterned nanofilm of material which is not specifically recognized by the bound functional moieties. It has been found that polyethylene oxide (PEO) can prevent such non-specific adsorption, as discussed, for example, in (1) Milthorpe, B. 2005. Protein adsorption to surfaces and interfaces, in: Surfaces and Interfaces for Biomaterials, Woodhead Publishing, 763-781. and (2) Frederix, F., Bonroy, K., Reekmans, G., Laureyn, W., Campitelli, A., Abramov, A., Dehaen, W., Maes, G. 2004. Reduced nonspecific adsorption on covalently immobilized protein surfaces using poly(ethylene oxide) containing blocking agents. J. Biochem. Biophys. Methods, 58, 67-74.

Thus, in at least one embodiment, the block copolymer comprising the present nanofilm or nanocoating can contain PEO as at least one hydrophilic block, and the proportion of PEO in the block copolymer can be advantageously selected to reduce or minimize non-specific adsorption to the resulting nanopatterned nanofilm or nanocoating. Without being bound by theory, it is considered that in such embodiments, the hydrophilic blocks 14, which are PEO blocks, can restrict non-specific adhesion at sufficient surface coverage. In such embodiments, it has been found that non-specific adsorption can be advantageously reduced or minimized when the transfer or deposit of the resulting nanopatterned nanofilm is carried out at surface pressures corresponding to the phase transition of PEO around the plateau of the π-A isotherm (about 10 mN/m).

Alternatively, or in addition, non-specific adsorption can be reduced or minimized by including in the nanofilm or nanocoating molecules containing short oligomeric ethylene oxide (EO) chains in addition to the present block copolymer. FIG. 3 is a diagrammatic representation of a surface 22 on which is adsorbed a functionalizable nanopatterned nanofilm containing an additional EO-containing molecule 26 in addition to the present modified block copolymer comprising the hydrophobic domains 20, hydrophilic blocks 14 and modifiable functional group 24. In at least one embodiment, the EO containing molecule contains no more than 20 ethylene oxide monomers. In at least one embodiment, the additional EO-containing molecule is H(—O—CH₂CH₂)_{z}S—S—(CH₂CH₂—O—)_{z}H (EO_{z}S—S-EO_{z}), where z is the number of ethylene oxide monomeric units. In at least one embodiment, 2z≤20. In at least one embodiment, the additional EO-containing molecule is H(—O—CH₂CH₂)₇—S—S—(CH₂CH₂—O—)₇H (EO₇—S—S-EO_{z}).

In at least one embodiment, the present functionalized nanofilms or nanocoatings are useful in biosensors. As is known in the art, biosensors are analytical devices that convert a biological response to a target substance (analyte) into a signal that measures a property of the analyte, such as its concentration. Recognition of the analyte by a biorecognition element, such as an antibody or enzyme, triggers a response at a detector which is converted into an easily measured signal which is then displayed. For example, surface plasmon resonance (SPR) chips can recognize analytes by detecting changes caused in the oscillation of conduction electrons at the analyte-detector interface.

In certain embodiments of the present functionalized nanopatterned nanofilms or nanocoatings which are useful for biosensor applications, the functional moiety present is a biorecognition element. As used herein, the term “biorecognition element” is intended to mean a moiety which can specifically recognize or be recognized by, and bind to, an analyte so as to be useful in a biosensor. Biorecognition elements can include but are not limited to naturally occurring or synthetic biomolecules and moieties which are specifically recognized by such naturally occurring or synthetic biomolecules. In at least one embodiment, the biorecognition element can include but is not limited to a nucleic acid, including but not limited to an aptamer or double-stranded or single-stranded DNA or RNA; a protein, including but not limited to an enzyme, an antibody, a lectin, or a receptor protein; or an antigen, or a small molecule which is specifically recognized by and binds to a biomolecule.

In certain embodiments which are useful for biosensor applications, the functionalized nanocoating is coated on a sensor surface which allows detection of a signal related to the interaction of an analyte with a functional moiety bound to the functionalized nanocoating. In certain embodiments, the sensor surface is compatible with and unharmed by conditions under which a functionalizable nanopatterned nanocoating coated thereon can be functionalized to attach a biorecognition element. Therefore, in at least one embodiment, the sensor surface can be coated with a functionalizable nanopatterned nanocoating as described herein, and the nanocoated sensor surface can be further functionalized with a biorecognition element to form a functionalized nanopatterned nanocoating, or nanobiocaptor. In at least one embodiment, the surface is a surface of a bioactive strip, a quartz crystal microbalance (QCM) chip or a surface plasmon resonance (SPR) chip. As is known in the art, a quartz crystal microbalance (QCM) measures mass per unit area by measuring the change in frequency of a quartz crystal resonator. Other suitable surfaces will be apparent to those skilled in the art. In at least one embodiment, the size of the hydrophobic and hydrophilic blocks selected to form the block copolymer comprising the nanocoating is selected such that the thickness of the resulting nanocoating does not prevent signal detection by the coated sensor surface.

The effectiveness of known biosensors can be compromised by factors which can affect detector sensitivity or create bias, such as uncontrolled receptor spatial density, mis-orientation of the biorecognition element, degradation of the biorecognition element following adsorption, and non-specific adsorption by interfering substances to the sensor surface. Additional challenges include the economic desire for a reversible reaction between the biorecognition element and the analyte, to allow multiple reuses of a biosensor. However, certain embodiments of the present functionalized nanopatterned nanofilm or nanocoating can provide features which may avoid or address one or more of the shortcomings of previously known biosensors.

In at least one embodiment, it is possible to control the biorecognition element coverage density on the biosensor surface by controlling the surface concentration, positioning and spacing of functional moieties attached to the present functionalized nanofilm or nanocoating coating the sensor surface, including but not limited to large functional moieties, such as biological molecules, as previously discussed. Thus, mis-orientation or steric hindrance of biorecognition elements immobilized on the nanofilm or nanocoating can be avoided and the sensitivity and accuracy of the biosensor can be optimized. In addition, in at least one embodiment, non-specific adsorption of possibly interfering moieties to the sensor surface can be reduced or minimized, as previously discussed, thereby allowing further optimization of biosensor sensitivity and specificity. Furthermore, at least in part as a result of the covalent linkage between the biorecognition element and at least one block copolymer of the nanopatterned nanocoating, certain embodiments of the present functionalized nanocoating may provide biosensors which are stable, robust and reusable and which are expected to have an advantageously long shelf life. In at least one embodiment, the present functionalizable and functionalized nanopatterned nanofilms or nanocoatings can be compatible with existing biosensor detectors and transducers.

As used herein, the terms “about” or “approximately” as applied to a numerical value or range of values are intended to mean that the recited values can vary within an acceptable degree of error for the quantity measured given the nature or precision of the measurements, such that the variation is considered in the art as equivalent to the recited values and provides the same function or result. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “substantially” refers to the complete or nearly complete extent or degree of an action, characteristic, property, state, structure, item, or result. For example, an object that is “substantially” aligned would mean that the object is either completely aligned or nearly completely aligned. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

### EXAMPLES

Other features of the present invention will become apparent from the following non-limiting examples which illustrate, by way of example, the principles of the invention.

### Example 1: Preparation of Maleimide-Modified PS-PEO Block Copolymer (PS-PEOm)

PS₁₈₃PEO₂₈₀ (Polymer Source, Montreal) is modified to add a terminal maleimide group using a Mitsunobu reaction.

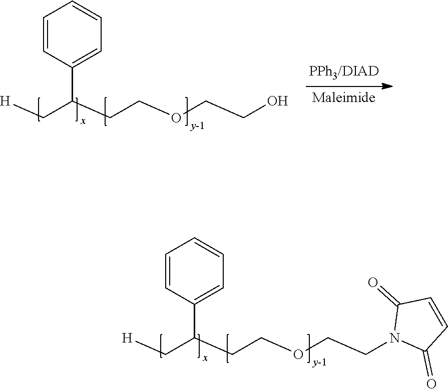

Triphenylphosphine (2 molar equivalents (2 eq)) is dissolved in benzene and cooled at 0° C. Diisopropyl azodicarboxylate (DIAD, 2 eq.) is added maintaining the temperature at 0° C. The mixture is stirred at 0° C. for 5 minutes then allowed to warm to room temperature (RT). PS₁₈₃PEO₂₈₀ (2 eq.) and tert-butanol (1 eq) are added and stirring is continued for 1 h at RT. The mixture is cooled to 0° C. and maleimide (1 eq) is added. The mixture is stirred for a further 5 minutes at 0° C., then allowed to warm to room temperature and stirring is continued overnight under argon atmosphere. For a preparation starting with 1 g of PS₁₈₃PEO₂₈₀ the mixture is concentrated to a volume of 5 mL and hexane is added to form a precipitate. The precipitate is filtered and dried under vacuum at room temperature. Formation of the maleimide derivative is confirmed by XPS, MS (MALDI-TOF) and by the appearance of a peak at 1723.4 cm⁻¹ (C═O) in the FT-IR spectrum of the product (maleimide-modified PS₁₈₃-PEO₂₈₀, also referred to herein as PS₁₈₃-PEO₂₈₀m).

### Example 2: Preparation of PS-PEO Coated Surface Plasmon Resonance (SPR) Chips

Mixtures of unmodified polystyrene-polyethylene oxide (PS₁₈₃-PEO₂₈₀) and varying percentages (0%, 0.1%, 1.3%, 6.0% and 11%) of PS₁₈₃-PEO₂₈₀ block co-polymers modified with maleimide (PS₁₈₃-PEO₂₈₀m) as described in Example 1 at a final concentration of approximately 1 mg/mL are prepared from solutions of PS₁₈₃-PEO₂₈₀ (1 mg/mL) and PS₁₈₃-PEO₂₈₀m (5 mg/mL), in spectra grade CHCl₃. The block copolymer solutions are prepared 24 h in advance. Gold-coated glass surfaces of surface plasmon resonance (SPR) chips (XanTec™) are cleaned using a UV/ozone cleaner (Novascan) for 1 h, followed by dipping in ethanol for 10 minutes, and are dried under a flow of argon gas.

Monolayers of the block copolymer mixtures are formed by spreading the block copolymers at the air-water interface using a microsyringe (Hamilton) and the Trurnit deposition method (Trurnit H J. A theory and method for the spreading of protein monolayers. Journal of Colloid Science. 1960; 15:1-13.). IUPAC recommendations were followed (Ter-Minassian-Saraga L, Catalysis CoCaSCl. Reporting experimental pressure-area data with film balances. Pure and Applied Chemistry. 1985; 57:621-32). The subphase was water with a resistivity of 18.2 MΩ-cm obtained by water purification using an Easypure™ II system (Barnstead/Thermolyne, Dubuque, Iowa, USA). The experiments were carried out at RT in a KSV2000 unit (KSV Instruments Ltd., Helsinki, Finland). The surface pressure was monitored with a platinum Wilhelmy plate. The system was placed on an antivibration table (System 63-541, TMC, MA, USA). Upon spreading, a waiting time of 30 minutes is allowed for solvent evaporation. Barrier speed was set at 10 mm/min and the surface pressure of transfer was fixed at 11 mN/m. The monolayers are transferred to the gold surfaces of clean SPR chips. After the Langmuir Schaefer transfer, the chips were dried under a flow of argon.

The XanTec™ chips coated with the block copolymer nanofilm were then further modified by dipping them into a solution of H(—O—CH₂CH₂)₇—S—S—(CH₂CH₂—O—)₇H (EO₇-S—S-EO₇; Polypure AS, Oslo, Norway) (1 mg/ml) in phosphate-buffered saline (PBS 100 mM pH 7), overnight. This procedure was also alternatively applied on the SPR chips directly in the SPR. Treatment of the chips with EO₇-S—S-EO₇ aids in preventing both non-specific adsorption and direct binding of cysteine-tagged proteins directly to the gold surface of the chip. This step allows for additional blocking of non-specific adsorption, and prevents binding of cys-tagged proteins directly on the gold surface.

### Example 3: Immobilization of Recombinant Protein G for Immunological Detection

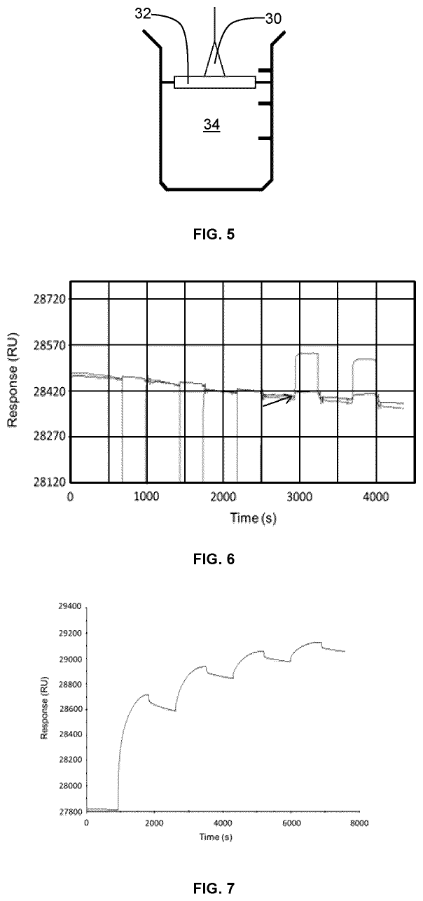

Coated SPR chips prepared as described in Example 2 are dipped in a solution of cysteine-tagged protein G (Prot G-Cys; 0.1 mg/mL) in PBS (pH 7) overnight at RT. A volume of 5 ml/chip was used. As shown diagrammatically in FIG. 5, suction device 30 or another suitable holder is attached to SPR chip 32 and used to dip SPR chip 32 in Prot G-Cys solution 34. The chips are then rinsed, dried under argon and fixed on the SPR chip holder using epoxy glue.

For the binding experiments, the coated SPR chip is inserted into an SPR instrument (Biacore X100 (GE Healthcare Life Sciences)) operated at 25° C. The chips are conditioned with phosphate-buffered saline (PBS) (100 mM) at pH 7 for 1 h under a flow rate of 10 μl/min. To ensure maximum binding of Prot G-Cys to the coated SPR chip, a solution of Prot G-Cys (0.1 mg/mL in PBS at pH 7) is injected until a stabilized signal is observed, indicating saturation. A solution of H—(O—CH₂CH₂-)₇SH (EO₇—SH; Polypure AS, Norway) (0.1 mg/mL) is then injected in order to react with any remaining unreacted maleimide groups and/or with any exposed Au surface. Rabbit immunoglobulin G (IgG; Sigma-Aldrich) is then injected in separate experiments. After allowing 10 minutes for binding, free IgG is removed by flushing the cell with buffer until a stable signal is achieved. Response is determined by subtracting baseline intensity from the intensity of the signal obtained at equilibrium. For all runs except IgG the flow rate was set at 10 μl/min; for IgG the flow rate was set at 5 μl/min.

An uncoated gold SPR chip is used as a control. Response is indicated in response units (RU). Au indicates the unmodified gold-coated SPR chip. EO₇—SH indicates H—(O—CH₂CH₂-)₇SH. The recombinant proteins G, with and without cysteine tags, were purchased from Biobasics (Markham Ontario, Canada). The rabbit immunoglobulin G (IgG) was obtained from Sigma-Aldrich. Typical results are presented in Table 1.

TABLE 1

| Entry | Surface | Elution | Response (RU) |
|---|---|---|---|
| A | Au | Prot G | 0 |
| B | Au | Prot G-Cys | 1660 |
| C | Au/PS₁₈₃-PEO₂₈₀ | (EO)₇-S-S-(EO)₇ | 70 |
| D | Au/PS₁₈₃-PEO₂₈₀/(EO)₇-S-S-(EO)₇ | Prot G-Cys | 0 |
| E | Au/PS₁₈₃-PEO₂₈₀-m (10%)/(EO)₇-S-S-(EO)₇ | Prot G-Cys | 1300 |
| F | Au/PS₁₈₃-PEO₂₈₀-m-Prot G-Cys (10%)/(EO)₇-S-S-(EO)₇ | (EO)₇-SH | 200 |
| G | Au/PS₁₈₃-PEO₂₈₀-m-Prot G-Cys-S-(EO)₇ (6%)/(EO)₇-S-S-(EO)₇ | IgG (0.01 mg/mL) | 240 ± 30 |
| | | IgG (0.1 mg/mL) | 1100 ± 1140 |
| H | Au-ProtG-Cys | IgG (0.01 mg/mL) | 23001 ± 200 |
| | | IgG (0.1 mg/mL) | 4500 ± 200 |

As indicated by entry A in Table 1 above, when an uncoated gold (Au) surface is exposed to protein G (Prot G), no significant binding is observed (0 response units (RU) are measured). However, exposing the uncoated Au surface to cysteine-tagged protein G (Prot G-Cys) shows a response of 1660 RU, indicating a high level of attachment (entry B). If an Au surface coated with a nanopatterned nanofilm prepared from the block copolymer PS₁₈₃-PEO₂₈₀ as described herein is exposed first to EO₇-S—S-EO₇ (entry C), then to Prot G-Cys (entry D), no significant binding is observed (0 RU). This indicates that the presence of the EO₇-S—S-EO₇ prevents non-specific binding of cysteine-tagged protein G to the surface. Without being bound by theory, it is believed that exposed Au sites on the surface are bound to EO₇-S—S-EO₇, thus preventing the non-specific as well as specific binding to gold via a cys-tag. The SPR traces obtained from the experiments of entries B and D are shown in FIG. 6. Addition of Prot G-Cys is indicated at the arrow. As seen in FIG. 6, the trace obtained from exposure of the uncoated Au surface to Prot G-Cys shows an increase in binding response, while the trace obtained from exposure to Prot G-Cys of the Au surface previously exposed to EO₇-S—S-EO₇ shows no change in binding response.

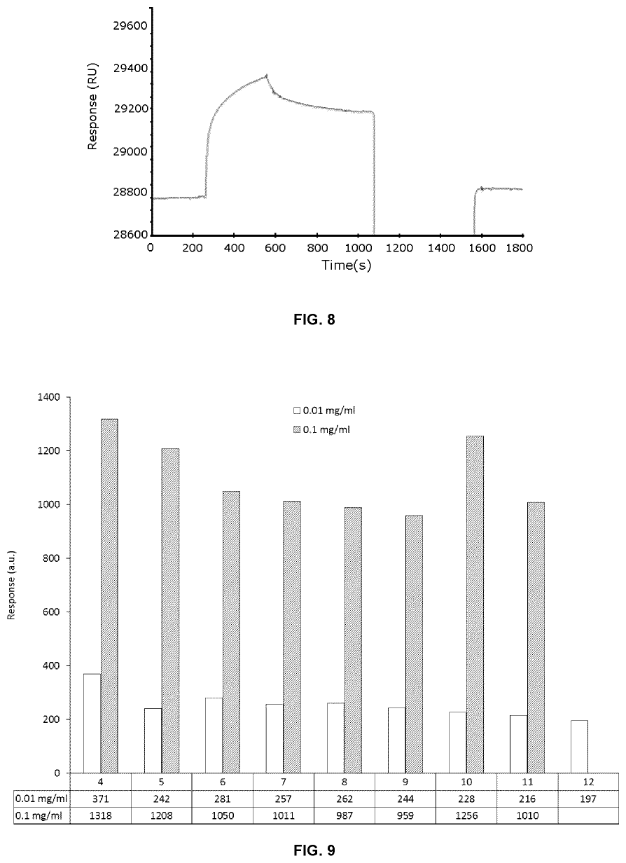

As indicated by entry E in Table 1, a gold surface coated with a functionalizable nanopatterned nanofilm prepared from the block copolymer PS₁₈₃-PEO₂₈₀ containing 10% PS₁₈₃-PEO₂₈₀-maleimide as described herein, in addition to EO₇-S—S-EO₇, shows specific binding of cysteine-tagged protein G (1300 RU) to the maleimide groups. The SPR sensorgram obtained from the experiment of entry E is shown in FIG. 7.

A nanobiocaptor surface specific for antibodies (IgG) can be prepared by coating a SPR chip with a functionalizable nanopatterned nanofilm prepared from the block copolymer PS₁₈₃-PEO₂₈₀ containing 10% PS₁₈₃-PEO₂₈₀-maleimide as described herein, in addition to EO₇-S—S-EO₇ (to inhibit binding), and treating the surface first with cysteine-tagged protein G (a biorecognition element which binds specifically to IgG antibodies, and which can covalently react with the maleimide groups present on the functionalizable nanopatterned nanocoating on the SPR chip, so as to covalently attach to the nanocoating), then with EO₇—SH. As indicated by entry F of Table 1, exposure of this surface to EO₇—SH causes further binding (200 RU), and it is believed that the treatment of the surface with EO₇—SH reacts with any maleimide groups remaining unreacted from the treatment with cysteine-tagged protein G, so as to prevent any further non-specific reaction with thiol-containing materials.

A nanobiocaptor surface prepared from a mixture of unmodified PS₁₈₃-PEO₂₈₀ containing 6% of PS₁₈₃-PEO₂₈₀m and further functionalized with protein G as described above is then exposed to an immunoglobulin G (IgG) preparation at two different concentrations (0.01 mg/mL and 0.1 mg/mL). Immunoglobulin G is known to bind specifically to protein G. As indicated by entry G in Table 1, significant binding to IgG was observed, even though only at most 6% of the surface was coated with the functionalizable PS₁₈₃-PEO₂₈₀m and could be bound to protein G. While a gold surface bearing covalently-bound Prot G-Cys showed higher binding to the IgG preparations, as indicated by entry H in Table 1, it is not possible to directly compare this to the results indicated by entry G, as the amount of protein G covalently bound to the gold surface is not known.

Moreover, the intensity of the SPR signal is known to decrease exponentially with the distance from the bare sensor surface (Erk T. Gedig. 2017. Surface Chemistry in SPR technology. Chap. 6, pp. 171-254 in Handbook of surface plasmon resonance, Edition 2. Richard B. M. Schasffort, editor, RSC Publishing). By using block copolymers with shorter blocks, such that the thickness of the resulting functionalizable or functionalized nanopatterned nanocoating is lower, SPR chips which are expected to provide higher intensity SPR signals can be prepared using the methods described herein. In the case of PS₁₈₃-PEO₂₈₀, the height of the dried domains (and thus the approximate thickness of the nanopatterned nanocoating) is in the range of 2-6 nm.

As can be seen from the SPR sensorgram presented in FIG. 8, antibodies (IgG) could be reversibly bound to SPR chips coated with coatings covalently modified with protein G. Exposure of a SPR chip surface coated with a nanopatterned nanocoating functionalized as described herein with protein G to an IgG preparation (0.01 mg/mL) shows binding, as evidenced by an increase in response. Washing the antibody-bound plate with glycine/HCl solution at pH 1.5 showed a drop in response, indicating removal of the IgG from the surface of the chip.

FIG. 9 shows the results of alternately exposing a SPR chip having a PS-PEO coating containing 6% PS-PEO modified with maleimide and protein G, as described above, with PBS containing IgG at concentrations of either 0.01 mg/mL or 0.1 mg/mL. The plate was washed by injection of glycine 10 mM (pH 1.5) for 5 min, PBS buffer pH 7 for 1 min, SDS 0.5% for 5 min, PBS buffer pH 7 for 1 min, water for 5 min, and PBS buffer pH 7 for 1 min to remove bound IgG between successive exposures to the IgG preparations. As seen from FIG. 9, the chip reliably shows a response reflecting the concentration of the IgG in the buffer, even after 12 successive trials, indicating that the plates can be re-usable.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples, but should be given the broadest interpretation consistent with the description as a whole.

1. A method of preparing a functionalizable nanopatterned nanocoating, the method comprising:
forming a nanopatterned nanofilm comprising one or more block copolymers,
wherein each of the one or more block copolymers comprises one or more hydrophobic blocks and one or more hydrophilic blocks, and
wherein at least one of the one or more block copolymers comprises at least one hydrophilic block which is terminated by a modifiable functional group; and
coating a substrate with the nanopatterned nanofilm to form the functionalizable nanopatterned nanocoating.

2. The method according to claim 1 wherein the hydrophobic block is selected from polystyrene and polybutadiene.

3. The method according to claim 2 wherein the hydrophobic block is polystyrene.

4. The method according to claim 1 wherein the hydrophilic block is polyethylene oxide.

5. The method of claim 1 wherein the modifiable functional group is a maleimide group.

6. The method of claim 1 wherein forming the nanopatterned nanofilm and coating the substrate with the nanopatterned nanofilm comprise using a Langmuir-Schaefer technique.

7. The method of claim 1 wherein the substrate is further coated with a molecule containing short oligomeric ethylene oxide chains operative to reduce non-specific adsorption to the substrate.

8. A method of preparing a functionalized nanopatterned nanocoating, the method comprising modifying the modifiable functional group of the nanopatterned functionalizable nanocoating according to claim 1 with a functional moiety.

9. The method of claim 8 wherein the modifiable functional group is a maleimide group and modifying the modifiable functional group with a functional moiety comprises reacting the maleimide group with a molecule containing a thiol group.

10. The method of claim 9 wherein the molecule containing a thiol group is a cysteine-tagged biomolecule.

11. The method of claim 10 wherein the cysteine-tagged biomolecule is a protein or a nucleic acid.

12. The method of claim 1 wherein the substrate is a sensor surface for a biosensor.

13. A functionalizable nanopatterned nanocoating formed by the method of claim 1.

14. A functionalized nanopatterned nanocoating formed by the method of claim 9.

15. A biosensor comprising a sensor surface coated with a functionalized nanopatterned nanocoating according to claim 14.
